(19) United States
(12) Patent Application Publication
(10) Pub. No.: US 2003/0114515 A1
(43) Pub. Date: Jun. 19, 2003

(54) Therapeutic mixture of HMG-COA reductase inhibitors

(76) Inventor: Wayne H. Kaesemeyer, Augusta, GA (US)

Correspondence Address: Raymond A. Miller, Pepper Hamilton LLP, One Mellon Center, 500 Grant Street, 50th Floor, Pittsburgh, PA, 15219, US

(21) Appl. No.: 10/269,847

(22) Filed: Oct. 11, 2002

Related U.S. Application Data
(63) Continuation of application No. 09/420,816, filed on Oct. 19, 1999, now abandoned.
Application No. 09/420,816 is a continuation-in-part of application No. 08/833,842, filed on Apr. 10, 1997, now Pat. No. 5,968,983.

(51) Int. Cl. A61K 31/401; A61K 31/366; A61K 31/198; A61K 31/225

(52) U.S. Cl. 514/423; 514/460; 514/548; 514/565

(57) ABSTRACT

Therapeutic mixtures of statins alone or in combination with L-arginine are described herein.

### BACKGROUND OF THE INVENTION

Recently it has been established that a family of enzymes called Nitric Oxide Synthase (“NOS”) form nitric oxide from L-arginine. The nitric oxide produced is linked to the endothelium dependent relaxation and activation of soluble guanylate cyclase, neurotransmission in the central and peripheral nervous systems, and activated macrophage cytotoxicity.

Nitric Oxide Synthase, occurs in many distinct isoforms. Formation of nitric oxide by the constitutive form (cNOS) in endothelial cells is thought to play an important role in normal blood pressure regulation, prevention of endothelial dysfunction such as hyperlipodemia, arteriosclerosis, thrombosis, and restenosis. The inducible form of nitric oxide synthase (iNOS) has been found to be present in activated macrophages and is induced in vascular smooth muscle cells, for example, by various cytokines and/or microbial products.

L-arginine is enzymatically converted into nitric oxide by NOS. Although initially described in endothelium, NOS activity has now been described in many cell types. Brain, endothelium, and macrophage isoforms appear to be products of a variety of genes that have approximately 50% amino acid identity. NOS in brain and in endothelium have very similar properties, the major differences being that brain NOS is cytosolic and the endothelial enzyme is mainly a membrane-associated protein.

### SUMMARY OF THE INVENTION

The term “subject” as used herein means any mammal, including humans, where nitric oxide (“NO”) formation from arginine occurs. The methods described herein contemplate prophylactic use as well as curative use in therapy of an existing condition.

The term “native NO” as used herein refers to nitric oxide that is produced through the bio-transformation of L-arginine or in the L-arginine dependent pathway. “EDRF” or “EDNO” may be used interchangeably with “native NO”. The term “endpoints” as used herein refers to clinical events encountered in the course of treating cardiovascular disease, up to and including death (mortality).

“L-arginine” as used herein includes all biochemical equivalents (i.e., salts, precursors, and its basic form). Bioequivalents of L-arginine include arginase inhibitors, lysine, citrulline, ornithine, and hydralazine (combinations of biochemical equivalents may also be employed).

“Agonist” refers to an agent which stimulates the bio-transformation of a NO precursor, such as L-arginine or L-lysine to EDNO or EDRF either through enzymatic activation, regulation or increasing gene expression (i.e., increased protein levels of c-NOS). Of course, either or both of these mechanisms may be acting simultaneously.

As used herein, the term “pharmaceutically acceptable carrier” refers to a carrier medium which does not interfere with the effectiveness of the biological activity of the active ingredients and which is not toxic to the hosts to which it is administered.

### BRIEF DESCRIPTION OF THE DRAWINGS

FIG. 1 is a schematic representation of NOS activation.

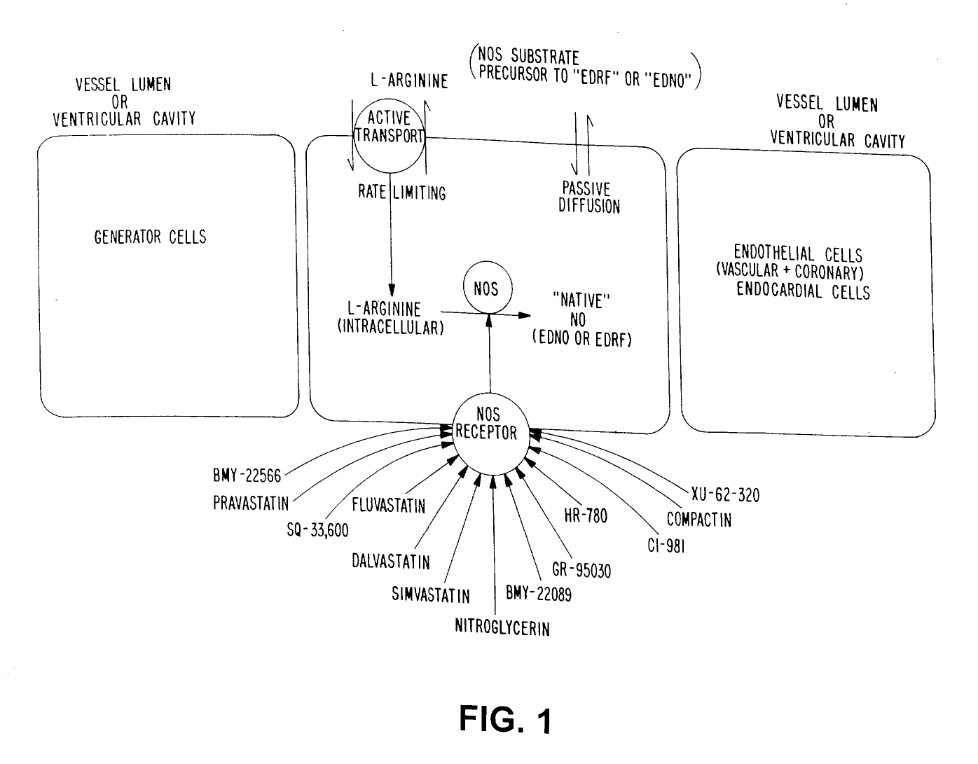

FIG. 2 is a bar graph illustrating the stimulation of NOS with pravastatin.

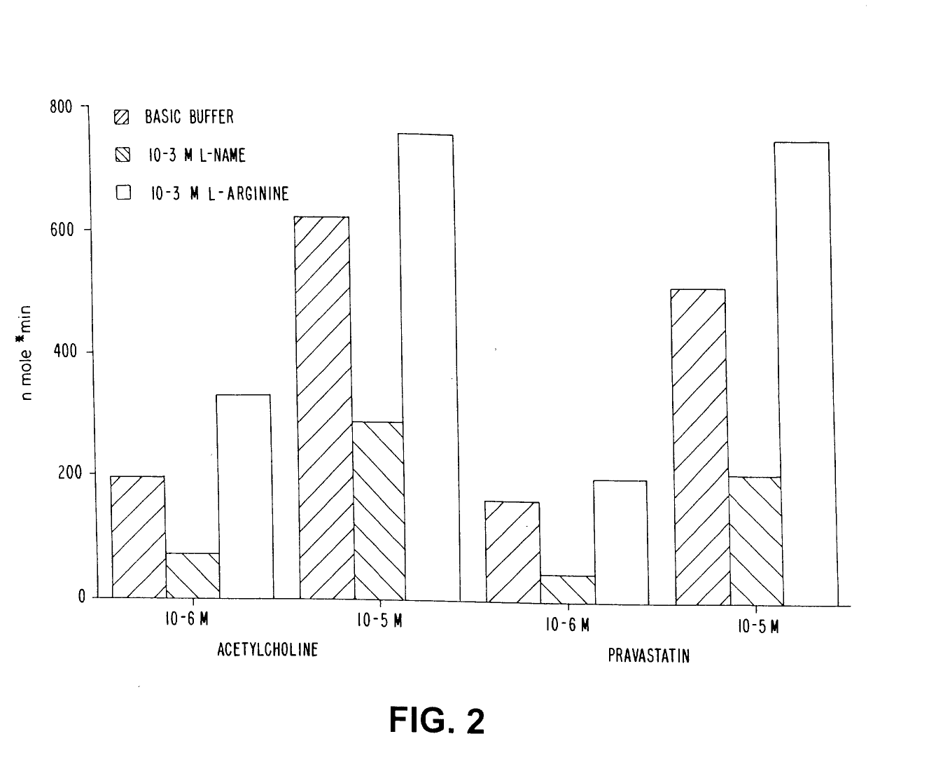

FIG. 3 is a schematic representation of the dynamics of L-arginine supply to NOS.

### DESCRIPTION OF THE SPECIFIC EMBODIMENTS

The present invention is preferably a combination of active ingredients, more preferably an agent that stimulates NOS activity and an agent which has another cardiocerebroreno vascular benefit. Even more preferably a substrate of NOS is employed in the mixture. Of particular interest as substrate are the amino acids, L-arginine and L-lysine, individually or in combination, as a mixture or as an oligopeptide, or a biologically equivalent compound, such as low molecular weight oligopeptides, having from about 2-10, usually 2-6 amino acids, or acetylated amino acids and oligopeptides, etc. An example of a biologically equivalent compound which appears to function as a substrate for NOS is L-arginine-L-phenylalanine and its methylester as is described in Timmerman et al., Br. J. Pharmacol. (1991), 104, 31-38 which is incorporated herein in its entirety by reference thereto.

The amount of the NO precursor agent, statin, or therapeutic mixture will be determined empirically in accordance with known techniques using animal models. The amount of the NO precursor agent (e.g., L-arginine) and/or statin employed preferably provide a physiologically effective amount to reduce proliferation of vascular smooth muscle cells and maintain the dilation of the vessel.

As used herein a “biological equivalent” is an agent or composition, or combination thereof, which has a similar biological function or effect as the agent or composition to which it is being deemed equivalent. For example, a biological equivalent of arginine is a chemical compound or combination of chemical compounds which has the same or similar biological function or effect as arginine. Lysine may be considered a biological equivalent of arginine. Other expected biological equivalents include citrulline, arginase inhibitors, hydralazine, and ornitine.

We originally discovered that dogs treated to a floor of nitroglycerin effect could be made further responsive by the co-administration of nitroglycerin and L-arginine in water in a manner similar to that commonly seen clinically with the addition of sodium nitroprusside (SNP) to nitroglycerin; however, when compared to SNP, L-arginine combined with nitroglycerin had much more favorable hemodynamic effects. Compared to SNP, vascular resistance was reduced by 50%, cardiac output doubled, and contractility increased. This led to the hypothesis that the combination of L-arginine and nitroglycerine was generating EDRF as opposed to SNP which is known to produce nitric oxide in a direct fashion. This is discussed in U.S. Pat. No. 5,543,430 and U.S. Pat. No. 5,767,160, both of which are incorporated by reference in their entirety.

As discussed in our U.S. Pat. No. 5,968,983, it appears that inhibitors of Hmg-CoA reductase may have dual applicability in the treatment of hypertension and cardiovascular diseases such that they act as both an inhibitor of the intrinsic biosynthesis of cholesterol and a stimulator or agonist of nitric oxide synthase. The fact that Hmg-CoA reductase may be agonist or stimulant of nitric oxide synthase has remarkable implications. We have shown that mixing inhibitors of Hmg-CoA reductase “in vitro” or “in vivo” with L-arginine has been found to have unforeseen beneficial effects (see e g., U.S. Pat. No. 5,968,983 which is hereby incorporated by reference in its entirety), L-arginine provides additional substrate for the Nitric Oxide Synthase and the NOS being catalyzed to enzymatically increase the bio-transformation of L-arginine into nitric oxide.

Virtually any of the family of those substances known as Hmg-CoA reductase inhibitors may be used in the present invention. These are taught for example in U.S. Pat. Nos. 4,857,522, 5,190,970, and 5,461,039, all of which are hereby incorporated by reference for this teaching. Those particular Hmg-CoA reductase inhibitors most preferred for use in conjunction with the present formulation as selected from the group consisting of: atorvastatin, cerivastatin, simvastatin, lovastatin, pravastatin, compactin, fluvastatin, and dalvastatin. U.S. Pat. No. 5,316,765 cites a number of these Hmg-CoA reductase inhibitors and is hereby incorporated by reference in its entirety. In particularly preferred embodiments of the present invention, the Hmg-CoA reductase inhibitor utilized is pravastatin or atorvastatin. In an even more particularly preferred embodiments, the administration of the present invention includes the Hmg-CoA reductase inhibitor pravastatin. These Hmg-CoA reductase inhibitors are commonly referred to as “statins.”

Not all hypercholesterolemic patients respond to statin treatment as currently known. There are patients who currently receive standard statin treatment but show no significant reductions in major coronary events. These patients and others would benefit from administration of a combination of two statins, one that is hydrophilic (soluble in water) for example, pravastatin, and one which is lipophilic or hydrophobic (insoluble in water) for example, atorvastatin. This dual statin treatment will be referred to as “combistatin” since it refers to administration of statins from two different categories.

While not wishing to be bound by theory, the rationale behind this treatment involves the differing modes of action of these statin agents. Both hydrophilic and hydrophobic statins, individually, are known to be effective in reducing total plasma and low density lipoprotein (LDL)-cholesterol concentrations and are associated with decreased heart-related mortality rates. The mixture of the present invention will reduce cholesterol by affecting two different pathways. Pravastatin (along with other hydrophilic statins) is believed to act principally outside of the liver. Therefore, the hydrophilic class of statins will exert their action primarily through increasing NO levels as an agonist of eNOS, due to increased exposure to the endothelium. The hydrophobic or lipophilic statins, such as atorvastatin, are fat soluble and are readily taken up by the liver and as a result, are effective in reducing cholesterol levels in the liver. It is believed that this cholesterol lowering activity is mediated by interactions of the hydrophobic statin with endothelial cells, which results in an inhibition of Hmg-CoA reductase activity and a concomitant upregulation of eNOS transcription and protein expression. As a result of these events in the liver, basal NO production is increased.

Thus, administration of the combination of a hydrophilic and a hydrophobic statin and optimally, with the NOS substrate L-arginine, is a method which will provide therapeutic treatment for cardiovascular disease which previously has been unresponsive to known statin therapy. Patients which will particularly benefit from such treatment include those with increased cholesterol levels and increased risk for cardiovascular events and whose cholesterol levels do not normalize with pravastatin and L-arginine therapy. These patients would then be administered a combination of e.g., pravastatin; a fat soluble statin (such as atorvastatin, lovastatin, simvastatin, cerivastatin, fluvastatin, dalvastatin and compactin); and optimally, L-arginine. The administration of a lipophilic statin in addition to pravastatin is more effective since the lipophilic, or fat soluble statin is readily taken up by the liver and will be more effective at reducing the cholesterol level than adding 40 mg more pravastatin. The total statin dose administered in the combistatin treatment should generally be within the safe range of 80 mg/day with 40 mg acting to increase NO levels and the other 40 mg acting to decrease the cholesterol that is not responding to pravastatin alone.

Where the particular Hmg-CoA reductase inhibitor is pravastatin, the ratio of pravastatin to atorvastatin is preferably within the range of 1:2 to 1:50, wt/wt. For example, pravastatin/atorvastatin at a ratio of 1:2 would include 40 mg/day pravastatin with 80 mg/day atorvastatin. Where the ratio of pravastatin/atorvastatin is at a ratio of 1:20, for example, 20 mg/day pravastatin would be administered with 400 mg/day atorvastatin. Weight ratio of ingredients described herein in regard to the Hmg-CoA reductase inhibitors, lovastatin, pravastatin and atorvastatin are found to be effective, however, each route of administration (e.g. IV, oral, transdermal, etc.) will vary in their requirements.

Even more particularly, the presently disclosed “mixtures” may be described in terms of their relative concentrations (grams) administered as part of a continuous daily and/or monthly regimen. In one particular embodiment, the formulation is administered so as to provide the patient with between 20-40 milligrams per day of the Hmg-CoA reductase inhibitor (e.g. pravastatin) together with a daily dose of atorvastatin of between 100 to 200 mg per day. Most preferably, the Hmg-CoA reductase inhibitor, such as pravastatin, is administered at a daily dose of about 20-40 mg per day together with a dose of about 40-80 mg per day atorvastatin. This particular embodiment of the claimed formulation should maintain within the patient efficient levels of the formulation.

Where the particular Hmg-CoA reductase inhibitor is pravastatin, the ratio of pravastatin to atorvastatin to L-arginine is preferably within the range of 1:1:1 to 1:50:50, wt/wt. For example, administration of pravastatin, atorvastatin and L-arginine at a ratio of 1:1:20 would include 40 mg/day pravastatin with 40 mg/day atorvastatin and 800 mg/day L-arginine. Weight ratio of ingredients described herein in regard to the Hmg-CoA reductase inhibitors, lovastatin, pravastatin and atorvastatin are found to be effective, however, each route of administration (e.g. IV, oral, transdermal, etc.) will vary in their requirements.

Even more particularly, the presently disclosed “mixtures” may be described in terms of their relative concentrations (grams) administered as part of a continuous daily and/or monthly regimen. In one particular embodiment, the formulation is administered so as to provide the patient with between 20-40 milligrams per day of the Hmg-CoA reductase inhibitor (e.g. pravastatin) together with a daily dose of atorvastatin of between 20-40 mg per day and a dose of Larginine of 100 to 200 mg per day. Most preferably, the Hmg-CoA reductase inhibitor, such as lovastatin, is administered at a daily dose of about 20 mg per day together with a dose of about 20 mg per day atorvastatin together with 200 mg per day dose of L-arginine. This particular embodiment of the claimed formulation should maintain within the patient efficient levels of the formulation.

It is likely that some cardiovascular and other diseases will benefit from treatment with any one or any combination of the following cholesterol lowering agents or inhibitors of cholesterol biosynthesis: ACE inhibitors, squalene synthetase inhibitors, fibric acid derivatives, bile acid sequestrants, MTP inhibitors, angiotensin receptor blockers, probucol, niacin and its biological equivalents, and isoprenoid phosphonates, In some instances it will be beneficial to utilize a mixture of any one or combination of the above agents with an HMG-CoA reductase inhibitor and L-arginine. Additionally, it may be beneficial to utilize a mixture of any one or combination of the above agents with L-arginine. There are a number of biological equivalents of L-arginine which can be used in place of L-arginine when this is beneficial to the patient, including L-lysine, arginase inhibitors, citrulline, ornithine, and hydralazine.

Even more particularly, the presently disclosed “mixtures” may be described in terms of their relative concentrations (grams) administered as part of a continuous daily and/or monthly regimen. In one particular embodiment, the formulation is administered so as to provide the patient with between 20-40 milligrams per day of the Hmg-CoA reductase inhibitor (e.g., pravastatin) together with a daily dose of L-arginine of between 100 to 200 mg per day. Most preferably, the Hmg-CoA reductase inhibitor, such as lovastatin, is administered at a daily dose of about 20 mg per day together with a dose of about 200 mg per day L-arginine. This particular embodiment of the claimed formulation should maintain within the patient efficient levels of the formulation.

The Hmg-CoA reductase inhibitors of the present invention are also characterized by an ability to stimulate receptor-mediated clearance of hepatic lowdensity lipoproteins (LDL), as an anti-hypercholesterolemic, and as a competitive inhibitor of Hmg-CoA reductase.

The preparation of lovastatin, simvastatin, and pravastatin have been described in the patent literature. The preparation of XU-62-320 (fluvastatin) is described in WIPO Patent WO84/02131. BMY 22089(13), CI 981(14), HR 780(15), and SQ 33,600 are also described in the literature cited, and are specifically incorporated herein by reference for the purpose of even more fully describing the chemical structure and synthesis of these Hmg-CoA reductase inhibitors. These methods of preparation are hereby incorporated by reference in their entirety.

Also within the scope of those Hmg-CoA reductase inhibitors of the present invention are included the bio-active metabolites of those Hmg-CoA reductase inhibitors described here, such as pravastatin sodium (the bio-active metabolite of mevastatin).

Any one or several of the Hmg-CoA reductase inhibitor compounds may be mixed with L-arginine or substrate precursor to endogenous nitric oxide to provide a therapeutically effective mixture. This therapeutically effective mixture can then be incorporated into a stent or other delivery device.

To demonstrate this, the direct effects of acteylcholine and pravastatin on NO production in bovine aortic endothelial cells (BAEC) was determined using a highly sensitive photometric assay for conversion of oxyhemoglobin to methemoglobin. NO oxidize; oxyhemoglobin (HbO₂) to methemoglobin (metHb) in the following reaction HbO₂+NO-metHb+NO₃. The amount of NO produced by endothelial cells was quantified by measuring the change in absorbance as HbO₂ oxidizes to metHb. Oxyhemoglobin has a absorbance peak at 415 nm, while metHb has a 406 nm absorbance peak. By subtracting the absorbance of methb from HbO₂, the concentration of NO can be assessed. The general method was patterned after that of Feelisch et al., (Biochem. and Biophy. Res. Comm. 1991; 180, Nc 1:286293).

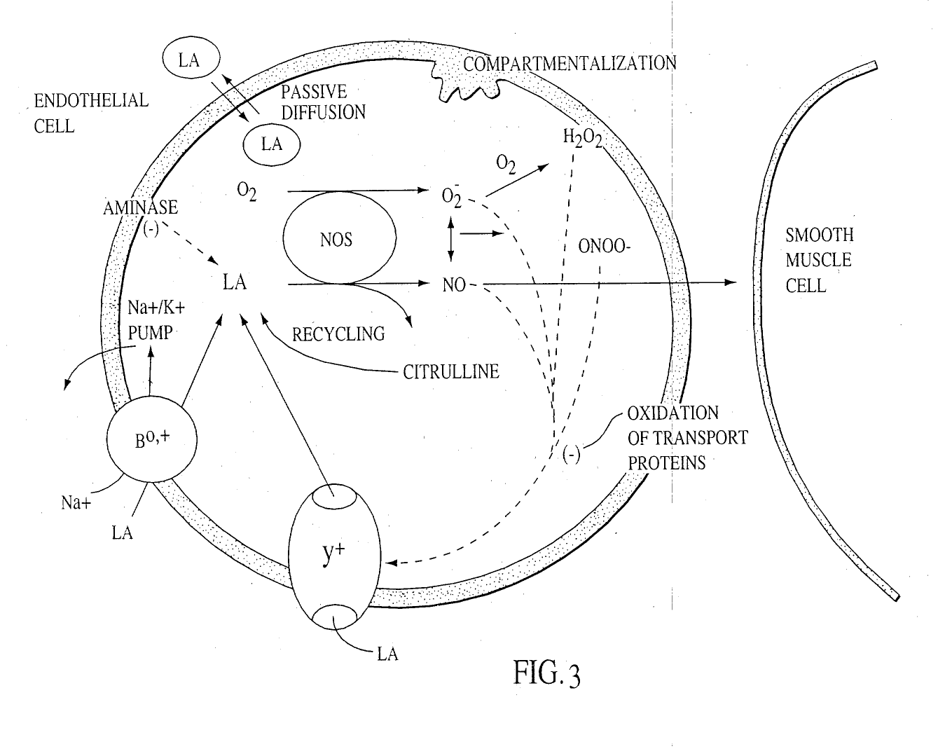

FIG. 3 is a bar graph of the data generated which illustrates the effects of acetylcholine and pravastatin (10⁻⁶ and 10⁻⁵ M) administered for 3 min periods into the cell/bead perfusion system on NO production with: 1) 10⁻⁵ M L-arginine in control (basic) buffer, 2) 10⁻³ M of L-NAME in buffer, and 3) 10⁻³ M of L-arginine in buffer. Responses are transient elevations in NO production above basal levels. Data for responses in L-NAME and L-arginine augmented buffer are presented as percent of response in control buffer (100%); numbers in basic buffer bars indicate absolute production of NO in nmole *min. The remaining two bars denote differences between responses in L-NAME buffer vs both basic and L-arginine added buffers.

The effects of pravastatin on activity of endothelial cells in producing NO were compared with those of actetylcholine, which is known to specifically stimulate NO production by NOS activity. Adding acetylcholine to the buffer superfusion bovine aortic endothelial cells (BAECs) grown on beads increased their production of NO as measured by oxidation of oxyhemoglobin to methemoglobin. Acetylcholine produced a transient, concentration-related increase in NO above baseline levels. In basic buffer containing 5×10⁻⁵M L-arginine, and there was approximately a two fold increase in NO production between 10⁻⁵ M L-arginine, there was approximately a two fold increase in NO production between 10⁻⁵ and 10⁻⁶ M acetylcholine. Subsequent treatment of these cells with buffer containing L-NAME, 10⁻³ M markedly reduced acetylcholine-induced production of NO by 80%. When this L-NAME buffer was replaced with another containing increased L-arginine (10⁻³ M), acetylcholine-elicited production of NO returned to control levels.

Pravastatin also caused a concentration-related increase in NO production above baseline levels. There was a larger increment in response to the 10⁻⁵ M concentrations of pravastatin (˜3×) compared with that of acetylcholine. Superfusion of the cell suspension with L-NAME (10⁻³ M), also blunted NO production in response to pravastatin. This suggests that NO production is due at least in part to NOS activity. Subsequent perfusion of the cells with a buffer containing L-arginine 10⁻³ M resulted in a return in NO production to a level above the amount induced by the Pravastatin in control (basis) buffer. This restoration of response to Pravastatin after L-arginine addition was greater than that observed for acetylcholine. Administration of Pravastatin or acetylcholine into a perfusion system containing only beads without cells did not induce metHb/NO production.

Even more particularly, the presently disclosed “mixtures” may be described in terms of their relative concentrations (grams) administered as part of a continuous intracoronary, intra-arterial, intra-luminal, intramural, intravenous and intrapericardial infusions. In one particular embodiment, the formulation is administered as mixtures of enhancers of NO production (e.g., NOS agonist or Hmg-CoA reductase inhibitors) with other Hmg-CoA reductase inhibitors and/or L-arginine encased in liposomes so as to provide maximum retention time of the mixture in any given vascular bed being perfused by a catheter delivering the therapeutic mixture. In some cases the liposomes containing the mixture may also contain genetic material for transfection of the genetic material into the surrounding tissue of the vascular bed. In some cases pellets containing the aforementioned mixtures may be directly implanted into the myocardium at the time of coronary bypass graft surgery. In yet another case, a therapeutic mixture is repeatedly infused into the pericardial space via an indwelling infusion catheter.

Compositions of the present invention may be in the form of an agent(s) in combination with at least one other agent, such as stabilizing compound, which may be administered in any sterile, bio-compatible pharmaceutical carrier, including, but not limited to, saline, buffered saline, dextrose, and water. The compositions may be administered to a patient alone, or in combination with other agents, drugs or hormones. Pharmaceutically-acceptable carriers may also be comprised of excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.) hereby incorporated herein by reference in its entirety. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they can be placed in an appropriate container and labeled for treatment of an indicated condition. Such labeling would include amount, frequency, and method of administration.

FIG. 3 is a schematic representation of the hypothesized dynamics of L-arginine supply to NOS. L-arginine levels are maintained primarily through the activity of the carrier-mediated Na⁺-independent transporter, y⁺, while the Na⁺-dependent transporter, B^{α+}, and passive diffusion account for less than 15%. Concurrent transport of L-arginine to NOS may control NO production. However, L-arginine supply to NOS can be limiting due to compartmentalization within EC, arginase activity or utilization of L-arginine by NOS. We believe that NO and superoxide anion reduce the activity of the y⁺ transporter and also reduce L-arginine available for NOS. Collectively, summation of supply verses demand or availability of L-arginine to NOS will determine whether NO or superoxide anion are formed. Collectively, our findings suggest that although intracellular L-arginine levels far exceed the concentration of L-arginine required by NOS for NO production, the amount of L-arginine available for utilization by NOS can be insufficient especially in conditions of chronic eNOS stimulation. The explanation for this L-arginine paradox may be provided by the work of McDonald and colleagues. Using porcine pulmonary artery endothelial cells with antibodies specific for caveolin, eNOS and the y⁺ transporter, McDonald et al. demonstrated that all of these proteins are co-localized within the plasma membrane caveolae. This suggests that eNOS associated with this complex is sequestered from overall intracellular L-arginine and relies on the de novo transport of L-arginine into the cell via the y⁺ transporter within the caveolae for NO production. If the transporter becomes damages as seen with oxidation, L-arginine supply could immediately become limiting and may be the basis for endothelial dysfunction. In addition, this eNOS/y⁺ transporter-caveolae complex may explain why endothelial dysfunction is quickly reversed with increasing extracellular LA. Once the transporter is turned off, L-arginine concentration gradient increases and delivery of L-arginine into cells is shifted towards passive diffusion. Therefore, extracellular supplementation of L-arginine may be helpful in driving passive diffusion of L-arginine when the integrity of carrier-mediated transporters cannot be maintained.

We believe that concurrent L-arginine supply to NOS via system y⁺, independent of overall intracellular L-arginine, is important in establishing and maintaining vascular function. Factors including NOS agonists and NO itself appear to control y⁺ activity and the summation of these factors is important in determining NO and superoxide anion formation, both of which contribute to vascular dysfunction and disease.

Pravastatin functions as both a NOS agonist and as a Hmg-CoA reductase inhibitor. This, along with its hydrophilic/hydrophobic (as well as the other statin's hydrophobicity) nature, leads to the compositions described and claimed herein.

It will be apparent to one of ordinary skill in the art that many changes and modifications can be made thereto without departing from the spirit or scope of the appended claims.

What is claimed is:

1. A composition comprised of
a first Hmg-CoA reductase inhibitor; and
a second Hmg-CoA reductase inhibitor, said first and second Hmg-CoA reductase being different.

2. The composition of claim 1, wherein said first Hmg-CoA reductase inhibitor is hydrophilic.

3. The composition of claim 2, wherein said first Hmg-CoA reductase inhibitor is pravastatin.

4. The composition of claim 3, wherein said second Hmg-CoA reductase inhibitor is atorvastatin.

5. The composition of claim 1, wherein said second Hmg-CoA reductase inhibitor is hydrophobic.

6. The composition of claim 5, wherein said second Hmg-CoA reductase inhibitor is atorvastatin.

7. The composition of claim 1, wherein said first Hmg-CoA reductase inhibitor is pravastatin and said second Hmg-CoA reductase inhibitor is atorvastatin.

8. The composition of claim 7, further including a biological equivalent of L-arginine.

9. A therapeutic composition comprising:
a hydrophilic HMG-CoA reductase inhibitor, and;
a hydrophobic HMG-CoA reductase inhibitor.

10. The therapeutic composition of claim 9, wherein the hydrophilic HMG-CoA reductase inhibitor is pravastatin.

11. The therapeutic composition of claim 9, wherein the hydrophobic HMG-CoA reductase inhibitor is atorvastatin.

12. The therapeutic composition of claim 9, wherein the hydrophobic HMG-CoA reductase inhibitor is selected from the group consisting of lovastatin, simvastatin, cerivastatin, fluvastatin, dalvastatin, and compactin.

13. The therapeutic composition of claim 9, further comprising Larginine.

14. A method for treating cardiovascular disease in a mammal comprising:
administering a hydrophilic HMG-CoA reductase inhibitor to a patient in need of such treatment, and;
administering a lipophilic HMG-CoA reductase inhibitor to a patient.

15. The method of claim 14, further comprising administering L-arginine.

16. The method of claim 14, wherein said hydrophilic Hmg-CoA reductase inhibitor is a NOS agonist.

17. The method of claim 14, wherein said lipophilic Hmg-CoA reductase inhibitor is administered to treat hyperlipidemia.

18. The method of claim 15, wherein said L-arginine is a substrate of NOS.

19. The method of claim 18, wherein said hydrophilic Hmg-CoA reductase inhibitor is pravastatin.
The method of claim 19, wherein said lipophilic Hmg-CoA reductase inhibitor is atorvastatin.
